(19) United States
(12) Patent Application Publication
(10) Pub. No.: US 2004/0171559 A1
(43) Pub. Date: Sep. 2, 2004

(54) Protection of stem cells from cytotoxic agents by modulation of beta-catenin signaling pathways

(76) Inventors: Irving Weissman, Redwood City, CA (US); Tannishtha Reya, Chapel Hill, NC (US)

Correspondence Address: BOZICEVIC, FIELD & FRANCIS LLP, 200 MIDDLEFIELD RD, SUITE 200, MENLO PARK, CA, 94025, US

(21) Appl. No.: 10/729,548

(22) Filed: Dec. 5, 2003

Related U.S. Application Data
(60) Provisional application No. 60/431,655, filed on Dec. 6, 2002.

(51) Int. Cl. A61K 31/7076; A61K 31/7072; A61K 31/513; A61K 31/522; A61K 31/525

(52) U.S. Cl. 514/27; 514/50; 514/46; 514/251; 514/263.37; 514/269

(57) ABSTRACT

Reagents that block the extracellular activation of β-catenin are used to induce quiescence in normal stem cells, in order to reduce the killing of stem cells by anti-proliferative agents.

### BACKGROUND OF THE INVENTION

Cytotoxic agents used in the treatment of cancer, including chemotherapy and radiotherapy, are known to injure and kill cells of both tumors and normal tissues. The successful use of chemotherapy to treat cancer depends upon the differential killing of cancer cells compared to the side effects on normal tissues. Among the more profound side effects are the killing of cells in the gut epithelia, and in the bone marrow. The destruction of bone marrow cells can lead to deficiencies in a variety of blood cells, resulting in, for example, neutropenia, agranulocytosis, thrombocytopenia, pancytopenia, or aplastic anemia. Acute and chronic bone marrow toxicities are therefore major limiting factors in the treatment of cancer, and neutropenia is a common limiting factor in dose escalation. Repeated or high dose cycles of chemotherapy may be responsible for severe stem cell depletion leading to important long-term hematopoietic sequelea and marrow exhaustion.

A cell of critical importance for maintaining bone marrow function is the hematopoietic stem cell, which call has the capacity to repopulate all of the hematopoietic lineages. While hematopoietic stem cells are often quiescent in normal adults, the severe depletion of mature blood cells during chemotherapy may cause a greater number of HSC to enter the cell cycle and differentiate. The administration of agents such as G-CSF or GM-CSF has been found to mobilize HSC into the peripheral blood, however the majority of thus mobilized CD34⁺ cells are not quiescent. Paradoxically, this increase in cell cycle activity may act against the long term interests of the patient, because cytotoxic agents are primarily effective against proliferating cells. While quiescent cells show a degree of drug insensitivity relative to cycling cells and might persist at the end of chemotherapy, cycling HSC are more susceptible to cytotoxic agents.

In particular, antimetabolites and inhibitors of DNA topoisomerase II are relatively ineffective against quiescent cells. These drugs include the widely used agents doxorubicin and carboplatinum, which inhibit type 11 topoisomerase. Antimetabolite agents may include pyrimidine analogs; purine analogs, and folic acid analogs. For example, methotrexate is widely used as an immunosuppressant, as well as in the treatment of hyperproliferative disorders.

Prevention or protection from the side effects of cytotoxic agents would be a great benefit to cancer patients. The many previous efforts to reduce these side effects have been largely unsuccessful. For life-threatening side effects, efforts have concentrated on altering the dose and schedules of the chemotherapeutic agent to reduce the side effects. And efforts such as the use of factors like colony stimulating factor (CSF), granulocyte-macrophage-CSF (GM-CSF) or epidermal growth factor (EGF) to increase the number of normal cells in various tissues before the start of chemotherapy may not be associated with increased survival of cells following chemotherapy.

Despite advances in the field of chemotherapy, prior art methods have proven to be of limited utility in minimizing chemotherapy-induced hematopoietic stem cell and blood cell depletion. Thus, there is a need for improved therapeutic methods and pharmaceutical compositions for increasing stem cell survival following chemotherapy.

Related Publications

Wnt proteins are intercellular signaling molecules that regulate development in several organisms and contribute to cancer when dysregulated. While loss of Wnt activity can lead to profound developmental defects, overactivation of Wnt signaling can have potent oncogenic effects. Wnts act by binding the receptors of the Frizzled family (Bhanot et al. (1996) Nature 382:225-30) in association with the low-density lipoprotein receptor related proteins (LRP). In the absence of a Wnt signal, the serine/threonine kinase GSK-3β phosphorylates beta-catenin, targeting it for ubiquitination and degradation by proteosomes. Binding of Wnt proteins to their receptors leads to beta-catenin stabilization and accumulation in the cytosol (Willert & Nusse (1998) Curr Opin in Gen Dev 8:95-102). Beta-catenin can then translocate to the nucleus, where it binds to members of the LEF-1/TCF family of transcription factors and causes induction of target genes Eastman & Grosschedl (1999) Curr Opin Cell Biol 11:233-40).

The use of β-catenin in the expansion of stem cells is discussed in U.S. Pat. No. 6,465,249. The use of wnt to stimulate hematopoietic stem cells is proposed in U.S. Pat. No. 5,851,984.

### SUMMARY OF THE INVENTION

Methods and compositions are provided for the protection of stem cells from cytotoxic agents, particularly cytotoxic agents that target proliferating cells, e.g. chemotherapeutic agents. Protection is achieved by administration of a dose of a protective agent that is effective in blocking the activation of β-catenin in stem cells through extracellular signaling. This protective agent prevents the replication of normal, i.e. non-tumor, stem cells while it is present, but allows the resumption of proliferation when it is no longer present. Normal stem cells include hematopoietic stem cells (HSC), gut epidermal stem cells, neural stem cells, etc. It is shown herein that stem cells require extracellular wnt signaling for proliferation and thus are rendered quiescent by administration of agents that block extracellular wnt signaling.

Protective agents of interest interfere with the interaction between soluble, extracellular wnt proteins, and the frizzled receptors that are present on the surface of stem cells. Such agents include, without limitation, soluble frizzled polypeptides comprising the wnt binding domains; soluble frizzled related polypeptides; wnt specific antibodies; frizzled specific antibodies; and other molecules capable of blocking extracellular wnt signaling.

In one embodiment of the invention, the protective agents have specificity for wnt proteins that interact with stem cells, particularly hematopoietic stem cells. In another embodiment of the invention, the protective agents have specificity for frizzled proteins expressed on the surface of stem cells, particularly by hematopoietic stem cells. There is overlap in the specificity of wnt proteins and frizzled receptors, and in some embodiments of the invention, the protective agents broadly interacts with multiple wnt proteins. Methods are provided for screening agents in vivo and in vitro for efficacy as protective agents.

In one embodiment of the invention, β-catenin activation from extracellular signaling is temporarily blocked by administration of a protective agent, which administration is performed before or during administration of a cytotoxic agent that targets proliferating cells. Cytotoxic agents that target proliferating cells include chemotherapeutic drugs used in the treatment of cancer. In one aspect, the cytotoxic agent is an inhibitor of enzymes involved in DNA synthesis, e.g. topoisomerases; polymerases, etc. In another aspect, the cytotoxic agent is an analog of a metabolite, e.g. a purine, pyrimidine or folic acid analog. In another aspect of the invention, the cytotoxic agent is an immunosuppressive agent. In another aspect, the cytotoxic agent is an antimicrobial agent.

In another embodiment of the invention, β-catenin activation from extracellular signaling is temporarily blocked by administration of a protective agent, which administration is performed before or during administration of a cytotoxic agent that targets proliferating cells, wherein at the conclusion of the chemotherapy, a dose of wnt protein effective to overcome the temporary block of stem cell proliferation is administered.

### BRIEF DESCRIPTION OF THE DRAWINGS

FIGS. 1A-1D. Activated β-catenin promotes growth of HSCs in vitro and maintains the immature phenotype of HSCs in long-term cultures. HSCs were infected with activated β-catenin-IRES-GFP or control GFP retrovirus, and subjected to cell cycle analysis after 60 h. a, β-catenin-infected cultures display an increased number of blasting cells (right box, S/G2/M) compared with control. b, For long-term growth studies, 10,000 infected HSCs were plated in 1 ng ml⁻¹ SLF and monitored over 60 days. Results are from one of five experiments. c, Giemsa staining reveals myeloid characteristics in control cells and HSC morphology (high nucleus to cytoplasm ratio) in β-catenin-infected cells. d, Control cells (grey lines) are largely lineage-positive, whereas most β-catenin-infected cells (black lines) are lineage-negative (Lin⁻) or have low levels (left panel). β-catenin-infected Lin⁻ cells have characteristics of HSCs, including low Thy-1.1 (middle panel), and high c-Kit and Sca-1 (right panel).

FIGS. 2A-2E. HSCs respond to Wnt signaling in native bone marrow microenvironment. HSCs were infected with a lentiviral reporter containing either LEF-1/TCF binding sites linked to destabilized GFP (TOP-dGFP), or mutated LEF-1/TCF binding sites linked to destabilized GFP (FOP-dGFP). Infected HSCs were transplanted into three lethally irradiated recipient mice, and analyzed after 14 weeks. The data shown represent two independent experiments. a, b, GFP expression is shown in donor-derived (a) or host-derived (b) HSCs. c, d, Donor-derived HSCs carrying mutated LEF-1/TCF reporter (c) as well as the recipient mouse HSCs (d) are GFP negative. Expression of GFP in donor-derived Lin⁻ c-Kit⁺ Sca-1⁻ cells (non-HSCs) is shown by thin lines (a-d). e, HSCs infected with TOP-dGFP or TOP-GFP (a non-destabilized GFP) were stimulated in vitro with control medium or with 100 ng ml⁻¹ Wnt3a, and the extent of GFP expression measured.

FIGS. 3A-3E. Inhibition of Wnt signaling reduces growth of HSCs in vitro and inhibits reconstitution in vivo. a, HSCs (20 cells per well) were cultured for 60 h in medium containing mitogenic factors and either IgG-CRD or control IgG. b, HSCs were infected with virus encoding axin-IRES-GFP or GFP alone. Growth of infected HSCs in the presence of mitogenic factors was monitored over 60 h. c, The number of live cells was determined by propidium iodide staining. d, e, The development of HSCs in vivo was determined by injecting 1,000 control or axin-infected cells per mouse into groups of four lethally irradiated, allelically marked (Ly5.2) host mice along with 300,000 competing syngeneic bone marrow cells. Cells were isolated from peripheral blood and analyzed by flow cytometry after >10 weeks. Donor-derived (Ly5.1⁺) cells were monitored in the peripheral blood of hosts; analysis from a representative recipient and average reconstitution is shown.

FIGS. 4A-4C. HSCs expressing β-catenin upregulate HoxB4 and Notch1. a, Purified wild-type HSCs were infected with activated β-catenin-IRES-GFP or control vector-IRES-GFP, and infected cells sorted based on GFP expression at 48 h. The RNA isolated from these cells was reverse transcribed and expression of HoxB4 and Notch1 was analyzed by real-time PCR analysis. Results are averaged over five independent PCR reactions. b, c, Representative graphs of real-time PCR analysis demonstrating equal amounts of GADPH (b) and differential amounts of HoxB4 (c) products from β-catenin-transduced HSCs (solid line) and control-transduced HSCs (dashed line). RFU, relative fluorescence units.

FIGS. 5A-5D. Wild Type HSCs proliferate to purified Wnt3A. Purified wild type mouse bone marrow HSCs were sorted by FACS and plated at 5 or 10 cells/well into 60 well Terasaki plates. Cells were incubated in X-vivo 15 (Bio Whittaker), 10% FBS, 5×10⁻⁵M 2-Mercaptoethanol, and 1×10⁻⁴M random methylated beta-cyclodextrin (CTD, Inc.) in the presence of either purified Wnt3A (at approx. 100 ng/ml) plus SLF (10 ng/ml) or SLF (10 ng/ml) alone, as a control. (SLF dose required ranged from 7.5 ng/ml-100 ng/ml depending on mouse strain used). Cell growth was monitored over a period of 7-9 days in culture, and is shown as total cell response (A) and the average frequency of responding wells (B) representative of over 9 independent experiments. To determine phenotypic characteristics, cells were plated in bulk (3500 cells) in 96 well plates and incubated in the presence of purified or unpurified Wnt3A. After seven days in culture, a majority of cells treated with purified Wnt3A (at 100 ng/ml) were negative for lineage markers (solid line) while a majority treated with unpurified Wnt3A (calculated to be at 200 ng/ml in the medium) strongly upregulated Lineage markers (dashed line) (C). FACS analysis of the purified Wnt3A treated cells demonstrated that the lineage negative population was distributed into c-Kit⁺ and Sca-1⁺ HSCs and c-kit⁺ and Sca-1⁻ myeloid progenitors (D).

FIG. 6. IgG-CRD inhibits Wnt mediated beta-catenin stabilization. 50,000 L cells were plated in a 24-well plate and treated with Wnt3A alone or Wnt3A in the presence of IgG-CRD (1:1) or control IgG (1:1). 12 hours after stimulation, cells were harvested and lysed (0.5% NP-40+20 mM Tris-pH8.0+170 mM NaCl, 1 mM EDTA-pH8.0+1 mM DTT+0.2 mM Na₃VO₄+protease inhibitors) for 15 min. on ice. Soluble protein lysates were separated by SDS-PAGE and transferred to PVDF. Western blots were probed with anti-β-catenin (BD Transduction Laboratories) and anti-actin (Sigma) antibodies.

### DETAILED DESCRIPTION OF THE EMBODIMENTS

Methods and compositions are provided for the protection of normal stem cells from cytotoxic agents that target proliferating cells, by administering a protective agent that blocks the activation of β-catenin through extracellular wnt pathway signaling. Normal stem cells include hematopoietic stem cells (HSC), gut epidermal stem cells, neural stem cells, etc., where protection of HSC is of particular interest.

It is shown herein that proliferation of stem cells requires wnt signaling; and conversely, that stem cells can be prevented from proliferating by blocking extracellular wnt signaling. Stem cells, including HSC, express frizzled proteins on their surface, which are receptors for wnt, and which activate intracellular β-catenin. Wnt signaling plays diverse roles at many stages of development by regulating the stability of β-catenin. In the absence of an activating signal, cytoplasmic β-catenin is bound to a multi-protein β-catenin destruction complex that contains several proteins including Axin, APC, and glycogen synthase kinase-3 (GSK3), and it is constitutively phosphorylated at a cluster of Ser and Thr residues at its N-terminus by GSK3. Phosphorylated β-catenin is recognized by β-TrCP, a component of the SCF^{βTrCP} ubiquitin-protein ligase complex, and degraded by the ubiquitin-proteasome pathway. Wnt signaling disassembles the β-catenin destruction complex, which prevents the phosphorylation and subsequent ubiquitination of β-catenin, thus diverting β-catenin from the proteasome machinery. Accumulated β-catenin then enters the nucleus, binds to the LEF/TCF family transcription factors, and activates the expression of β-catenin target genes.

Unlike normal cells, many common tumor cells do not require extracellular wnt signaling for proliferation. Aberrant activation of the wnt signaling pathway, which can be the result of activating mutations of β-catenin or inactivating mutations of APC or Axin, has been associated with a wide variety of human malignancies, such as colorectal, heptocellular, ovarian endometrial, desmoid, leukemia (CML) and pancreatic tumors. For example, APC is mutated in the majority of colorectal cancers, and those tumors with wild-type APC often contain mutated β-catenin. Thus, aberrant activation of Wnt signaling is obligatory for the initiation or progression of colorectal tumors.

Protective agents of interest interfere with the interaction between soluble, extracellular wnt proteins, and frizzled proteins on the surface of stem cells. Such agents include, without limitation, soluble frizzled polypeptides comprising wnt binding domains; soluble frizzled related proteins; wnt specific antibodies and biologically active fragments thereof; frizzled specific antibodies and biologically active fragments thereof; and other molecules capable of blocking extracellular wnt signaling. The agents do not affect the proliferation of tumor cells, which therefore remain sensitive to anti-proliferative agents.

β-catenin activation from extracellular signaling may be temporarily blocked by administration of a protective agent before or during administration of a cytotoxic agent that targets proliferating cells. The methods of the invention are also particularly suitable for those patients in need of repeated or high doses of chemotherapy. For some cancer patients, hematopoietic toxicity frequently limits the opportunity for chemotherapy dose escalation. Repeated or high dose cycles of chemotherapy may be responsible for severe stem cell depletion leading to important long-term hematopoietic sequelea and marrow exhaustion. The methods of the present invention provide for improved mortality and blood cell count when used in conjunction with chemotherapy.

The methods, kits, and pharmaceutical compositions of the present invention, by increasing stem cell survival following chemotherapy significantly enhance the utility of presently available treatments for clinical chemotherapeutic treatments.

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Frizzled polypeptides and soluble frizzled polypeptides. Members of the ‘frizzled’ (Fz) gene family encode 7-transmembrane domain proteins that are receptors for Wnt signaling proteins. Among the human Fzd gene family are FZD1-10. FZD1, 3, 4, 6, 7 and 8 are of particular interest. Hematopoietic stem cells have been reported to express, inter alia, FZD4 (see Natalia et al. (2002) Science 298(5593): 601-604). Among frizzled proteins, the cysteine rich domain (CRD) contains the wnt-binding determinants, and is both necessary and sufficient for conferring wnt binding to transfected cells. Soluble FZD CRDfind use as inhibitors of extracellular wnt signaling, in particular the CRD of FZD8 interacts with a broad spectrum of wnt proteins. FZD proteins also find use as an immunogen for raising blocking antibodies.

The predicted 647-amino acid FZD1 protein contains a signal peptide, a cysteine-rich domain in the N-terminal extracellular region, 7 transmembrane domains, and a C-terminal PDZ domain-binding motif. FZD1 shares 77% and 74% protein sequence identity with FZD2 and FZD7, respectively. FZD1 has the Genbank accession number AB017363; (Sagara et al. (1998) Biochem. Biophys. Res. Commun. 252 (1), 117-122). FZD2 has the Genbank accession number AB017364; (Sagara et al. supra).

The 666-amino acid FZD3 protein, which is 98% identical to mouse Fzd3, contains an N-terminal CRD, 7 transmembrane domains, 2 cysteine residues in the second and third extracellular loops, and 3 N-linked glycosylation sites. Northern blot analysis revealed expression of 14.0-, 9.0-, 4.0-, and 1.8-kb FZD3 transcripts mostly in central nervous system (CNS) tissue, in adult pancreas and in many cancer cell lines. FZD3 has Genbank accession number AJ272427, (Kirikoshi et al. (1999) Biochem. Biophys. Res. Commun. 264 (3), 955-961).

FZD4 encodes a deduced 537-amino acid protein that has a cysteine-rich domain in the N-terminal extracellular region, 2 cysteine residues in the second and third extracellular loops, 2 N-linked glycosylation extracellular sites, and the S/T-X-V motif in the C terminus. Northern blot analysis indicates expression of a 7.7-kb transcript in large amounts in adult heart, skeletal muscle, ovary, and fetal kidney; in moderate amounts in adult liver, kidney, pancreas, spleen, and fetal lung; and in small amounts in placenta, adult lung, prostate, testis, colon, fetal brain, and liver. FZD4 has the Genbank accession number AB032417; (Kirikoshi et al. (1999) Biochem. Biophys. Res. Commun. 264 (3), 955-961

FZD5 encodes a polypeptide of a polypeptide of 585 amino acids, which is reported to be a receptor for Wnt5A. FZD5 has the Genbank accession number AB043702.

The predicted 706-amino acid FZD6 protein contains a signal peptide, a cysteine-rich domain in the N-terminal extracellular region, and 7 transmembrane domains. However, unlike many other Fz family members, FDZ6 does not contain a C-terminal PDZ domain-binding motif. FZD6 has the Genbank accession number AB012911; (Tokuhara et al. (1998) Biochem. Biophys. Res. Commun. 243 (2), 622-627).

The predicted 574-amino acid FZD7 protein contains an N-terminal signal sequence, 10 cysteine residues typical of the cysteine-rich extracellular domain of Fzd family members, 7 putative transmembrane domains, and an intracellular C-terminal tail with a PDZ domain-binding motif. FZD7 has the Genbank accession number AB017365; (Sagara et al. (1998) Biochem. Biophys. Res. Commun. 252 (1), 117-122).

FZD8 is a 694-amino acid protein, which is 69% identical to FZD5 and 95% identical to mouse Fzd8, contains an N-terminal signal peptide, a CRD, 7 transmembrane domains, 3 N-linked glycosylation sites, and a C-terminal ser/thr-X-val motif, which is a binding site for scaffold proteins with multiple PDZ domains. A 4.0-kb FZD8 transcript is most abundant in fetal kidney, followed by fetal brain and fetal lung. In adult tissue, FZD8 is expressed in kidney, heart, pancreas, and skeletal muscle. FZD8 has the Genbank accession number AB043703; (Saitoh et al. (2001) Int. J. Oncol. 18 (5), 991-996). FZD9 has the Genbank accession number BC026333; (Strausberg et al. (2002) Proc. Natl. Acad. Sci. U.S.A. 99 (26), 16899-16903).

FZD10 is a 581-amino acid protein, which is 66% identical to FZD9, contains an N-terminal CRD; 7 transmembrane domains with 2 cysteine residues in the second and third extracellular loops; 2 N-linked glycosylation sites; and a C-terminal ser/thr-Xxx-val motif, which is a binding site for scaffold proteins with multiple PDZ domains. It is widely expressed, with highest levels in placenta and fetal kidney, followed by fetal lung and brain. Within adult brain, expression was relatively high in cerebellum, followed by cerebral cortex, medulla, and spinal cord. FZD10 has the Genbank accession number AB027464; (Koike et al. (1999) Biochem. Biophys. Res. Commun. 262 (1), 39-43.)

Each frizzled protein contains at its amino terminus a conserved, extracellular cysteine rich domain, which spans approximately 120 amino acids and contains 10 invariant cysteines, followed by 7 membrane spanning domains. For use in the methods of the invention, soluble forms of the CRD are of interest. Such domains are characterized as retaining the wnt binding capability of the molecule, and will generally include the invariant cysteine residues, but will lack the membrane spanning domains. Examples of CRD constructs may be found, for example, in Hsieh et al. (1999) PNAS 96:3546-3551, herein incorporated by reference.

Frizzled related proteins. The secreted frizzled-related proteins (sFRPs) are approximately 30 kDa in size, and each contains a putative signal sequence, a cysteine-rich domain of approximately 110 residues that is 30 to 40% identical to the putative ligand-binding domain of FZ proteins, but lacks the 7-transmembrane motif that anchors FZ proteins to the plasma membrane, and conserved hydrophilic carboxy-terminal domain. FRP is secreted but, like wnt, tends to remain associated with cells. When coexpressed with various wnt family members, FRP antagonizes wnt-dependent activity, behaving like a dominant-negative receptor. FRP proteins are therefore inhibitors of wnt, and act to bind soluble wnt, thereby blocking activation through the membrane-bound frizzled protein.

Human SFRP1 contains 314 amino acids. The sequence may be found at Genbank, accession number AF001900, and is described by Finch et al. (1997) P.N.A.S. 94(13):6770-6775.

SFRP2 is expressed as 2.2- and 1.3-kb transcripts in several human tissues, with the highest levels in colon and small intestine. The sequence may be found at Genbank, accession number AY359001, and is described by Clark et al. (2003) Genome Res. 13 (10), 2265-2270.

SFRP3 contains a 25-amino acid signal peptide, an N-terminal N-glycosylation site, a 24-amino acid putative transmembrane segment, a region with multiple potential ser/thr phosphorylation sites, and a serine-rich C-terminal domain. The sequence may be found at Genbank, accession number U24163; Hoang et al. (1996) J. Biol. Chem. 271 (42), 26131-26137.

The 346-amino acid SFRP4 protein contains an N-terminal signal peptide, no transmembrane domain, and a hydrophilic C terminus. In situ hybridization analysis demonstrated exclusive expression in stromal and myometrial cells, particularly in endometrium and breast. The sequence may be found at Genbank, accession number AF026692.

SFRP5 is highly expressed in the retinal pigment epithelium (RPE). Like other SFRPs, SFRP5 contains an N-terminal signal peptide followed by a region homologous to the frizzled cysteine-rich domain (CRD). The sequence may be found at Genbank, accession number AF117758, and is described by Chang et al. (1999) Hum. Mol. Genet.

Wnt polypeptides. As used herein, the terms “Wnts” or “Wnt gene product” or “Wnt polypeptide” refers to members of the Wnt gene family. Included in the designation are human Wnt polypeptides. Human wnt proteins include the following: Wnt 1, Genbank reference NP_{—}005421.1; Wnt 2, Genbank reference NP_{—}003382.1, which is expressed in brain in the thalamus, in fetal and adult lung and in placenta; two isoforms of Wnt 2B, Genbank references NP_{—}004176.2 and NP_{—}078613.1. Isoform 1 is expressed in adult heart, brain, placenta, lung, prostate, testis, ovary, small intestine and colon. In the adult brain, it is mainly found in the caudate nucleus, subthalamic nucleus and thalamus. Also detected in fetal brain, lung and kidney. Isoform 2 is expressed in fetal brain, fetal lung, fetal kidney, caudate nucleus, testis and cancer cell lines. Wnt 3 and Wnt3A play distinct roles in cell-cell signaling during morphogenesis of the developing neural tube, and have the Genbank references NP_{—}110380.1 and X56842. Wnt3A is expressed in bone marrow. Wnt 4 has the Genbank reference NP_{—}110388.2. Wnt 5A and Wnt 5B have the Genbank references NP_{—}003383.1 and AK013218. Wnt 6 has the Genbank reference NP_{—}006513.1; Wnt 7A is expressed in placenta, kidney, testis, uterus, fetal lung, and fetal and adult brain, Genbank reference NP_{—}004616.2. Wnt 7B is moderately expressed in fetal brain, weakly expressed in fetal lung and kidney, and faintly expressed in adult brain, lung and prostate, Genbank reference NP_{—}478679.1. Wnt 8A has two alternative transcripts, Genbank references NP_{—}114139.1 and NP_{—}490645.1. Wnt 8B is expressed in the forebrain, and has the Genbank reference NP_{—}003384.1. Wnt 10A has the Genbank reference NP_{—}079492.2. Wnt 10B is detected in most adult tissues, with highest levels in heart and skeletal muscle. It has the Genbank reference NP_{—}003385.2. Wnt 11 is expressed in fetal lung, kidney, adult heart, liver, skeletal muscle, and pancreas, and has the Genbank reference NP_{—}004617.2. Wnt 14 has the Genbank reference NP_{—}003386.1. Wnt 15 is moderately expressed in fetal kidney and adult kidney, and is also found in brain. It has the Genbank reference NP_{—}003387.1. Wnt 16 has two isoforms, Wnt-16a and Wnt-16b, produced by alternative splicing. Isoform Wnt-16B is expressed in peripheral lymphoid organs such as spleen, appendix, and lymph nodes, in kidney but not in bone marrow. Isoform Wnt-16a is expressed at significant levels only in the pancreas. The Genbank references are NP_{—}057171.2 and NP_{—}476509.1.

While methods of in vivo treatment are typically directed at native, or naturally occurring Wnt polypeptides; for in vitro screening purposes, Wnt polypeptide variants, Wnt polypeptide fragments and chimeric Wnt polypeptides may find use. A “native sequence” polypeptide is one that has the same amino acid sequence as a Wnt polypeptide derived from nature. The native sequence of human Wnt polypeptides may range from about 348 to about 389 amino acids long in their unprocessed forms, reflecting variability at the poorly conserved amino-terminus and several internal sites, contain 21 conserved cysteines, and have the features of a secreted protein. The molecular weight of a Wnt polypeptide is usually about 38-42 kD.

Wnt inhibitor. For the purposes of the present invention, wnt inhibitors are agents that block the interaction between extracellular wnt protein and the cognate frizzled receptor on stem cells; and are used as a stem cell protective agent in the methods of the invention. Agents of interest may interact directly with a specific wnt, a specific set of wnts, or broadly with wnt proteins. Other agents of interest may interact directly with a specific frizzled, a specific set of frizzled proteins, or broadly with frizzled proteins. Agents of interest include blocking antibodies; or biologically active fragments thereof, e.g. Fv fragments, FAb fragments, and the like. Other inhibitors of interest interact with wnt-associated proteins, e.g. Wnt co-receptors LRP5/6 and the transmembrane protein Kremen.

Inhibitors of interest interfere with the frizzled and/or wnt proteins that interact with stem cells, particularly hematopoietic stem cells. Such cells have been reported to express FZD4; and wnt10A (Natalia et al. (2002) supra). HSC are also shown herein to be responsive to wnt 3A. Stromal cells in the bone marrow, which produce factors active on HSC, have been reported to express Wnt 2B; Wnt 10B and Wnt 5A.

A number of wnt inhibitors have been described and are known in the art. Among the known wnt inhibitors are members of the Dickkopf (Dkk) gene family (see Krupnik et al. (1999) Gene 238(2):301-13). Members of the human Dkk (“hDkk”) gene family include Dkk-1, Dkk-2, Dkk-3, and Dkk-4, and the Dkk-3 related protein Soggy (Sgy). hDkks 1-4 contain two distinct cysteine-rich domains in which the positions of 10 cysteine residues are highly conserved between family members. Exemplary sequences of human Dkk genes and proteins are publicly available, e.g. Genbank accession number NM_{—}014419 (soggy-1); NM_{—}014420 (DKK4); AF177394 (DKK-1); AF177395 (DKK-2); NM_{—}015881 (DKK3); and NM_{—}014421 (DKK2).

Other inhibitors of wnt include Wise (Itasaki et al. (2003) Development 130(18):4295-30), which is a secreted protein. The Wise protein physically interacts with the Wnt co-receptor, lipoprotein receptor-related protein 6 (LRP6), and is able to compete with Wnt8 for binding to LRP6. Axin regulates Wnt signaling through down-regulation of beta-catenin (see Lyu et al. (2003) J Biol Chem. 278(15):13487-95).

Soluble forms of the ligand binding domain (CRD) of Frizzled inhibit wnt; as do the soluble frizzled related proteins described above (Krypta et al, J Cell Sci 2003 July 1; 116(Pt 13):2627-34). The Frizzled-CRD domain has been shown to inhibit the Wnt pathway by inhibiting the binding of Wnts to the frizzled receptor (Hsieh et al. (1999) Proc Natl Acad Sci U S A 96:3546-51; and Cadigan et al. (1998) Cell 93:767-77).

The FZD8 CRD has been used as an inhibitor because of its broad binding spectrum against wnt proteins; although other CRDs also find use. The CRD may be fused to another polypeptide to provide for added functionality, e.g. to increase the in vivo stability. Generally such fusion partners are a stable plasma protein that is capable of extending the in vivo plasma half-life of the CRD when present as a fusion, in particular wherein such a stable plasma protein is an immunoglobulin constant domain.

In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the CRD also will be produced and employed as a multimer having substantially the same structure as the stable plasma protein precursor. These multimers will be homogeneous with respect to the CRD they comprise, or they may contain more than one CRD.

Stable plasma proteins are proteins typically having about from 30 to 2,000 residues, which exhibit in their native environment an extended half-life in the circulation, i.e. greater than about 20 hours. Examples of suitable stable plasma proteins are immunoglobulins, albumin, lipoproteins, apolipoproteins and transferrin. The CRD typically is fused to the plasma protein at the N-terminus of the plasma protein or fragment thereof which is capable of conferring an extended half-life upon the CRD. Increases of greater than about 100% on the plasma half-life of the CRD are satisfactory.

Ordinarily, the CRD is fused C-terminally to the N-terminus of the constant region of immunoglobulins in place of the variable region(s) thereof, however N-terminal fusions may also find use. The transmembrane regions or lipid or phospholipid anchor recognition sequences of frizzled proteins are preferably deleted prior to fusion.

Typically, such fusions retain at least functionally active hinge, CH2 and CH3 domains of the constant region of an immunoglobulin heavy chain, which heavy chains may include IgG1, IgG2a, IgG2b, IgG3, IgG4, IgA, IgM, IgE, and IgD, usually one or a combination of proteins in the IgG class. Fusions are also made to the C-terminus of the Fc portion of a constant domain, or immediately N-terminal to the CH1 of the heavy chain or the corresponding region of the light chain. This ordinarily is accomplished by constructing the appropriate DNA sequence and expressing it in recombinant cell culture. Alternatively, the polypeptides may be synthesized according to known methods.

The precise site at which the fusion is made is not critical; particular sites are well known and may be selected in order to optimize the biological activity, secretion or binding characteristics of the CRD. The optimal site will be determined by routine experimentation.

In some embodiments the hybrid immunoglobulins are assembled as monomers, or hetero- or homo-multimers, and particularly as dimers or tetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of basic four-chain units held together by disulfide bonds. IgA immunoglobulin, and occasionally IgG immunoglobulin, may also exist in a multimeric form in serum. In the case of multimers, each four chain unit may be the same or different.

Inhibitors useful in this invention also include derivatives, variants, and biologically active fragments of naturally occurring inhibitors, antibodies, and the like. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%.

A “chimeric” polypeptide is a polypeptide comprising a polypeptide or portion (e.g., one or more domains) thereof fused or bonded to heterologous polypeptide. A chimeric frizzled protein, for example, will share at least one biological property in common with a native sequence frizzled polypeptide. Examples of chimeric polypeptides include immunoadhesins, as described above, which combine a portion of the frizzled polypeptide with an immunoglobulin sequence, and epitope tagged polypeptides, which comprise a frizzled polypeptide or portion thereof fused to a “tag polypeptide”. The tag polypeptide has enough residues to provide an epitope against which an antibody can be made, yet is short enough such that it does not interfere with biological activity of the frizzled polypeptide. Suitable tag polypeptides generally have at least six amino acid residues and usually between about 6-60 amino acid residues.

A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. The term “derivative” encompasses both amino acid sequence variants of polypeptide and covalent modifications thereof.

Suitable wnt inhibitors may be identified by compound screening by detecting the ability of an agent to affect the biological activity of wnt. In vitro assays may be conducted as a first screen for efficacy of a candidate inhibitor, and usually an in vivo assay will be performed to confirm the biological assay. Desirable inhibitors are effective in temporarily blocking wnt signaling, and concurrent stem cell proliferation, but do not cause the death of stem cells during the blocking period. Desirable inhibitors are temporary in nature, e.g. due to biological degradation; or may be followed by administration of a wnt protein to “wash out” the inhibitor.

In vitro assays for wnt biological activity include, e.g. stabilization of β-catenin, promoting growth of stem cells, etc. Assays for biological activity of Wnt include stabilization of β-catenin, which can be measured, for example, by serial dilutions of the Wnt composition. An exemplary assay for Wnt biological activity contacts a Wnt composition in the presence of a candidate inhibitor or activator with cells, e.g. mouse L cells. The cells are cultured for a period of time sufficient to stabilize β-catenin, usually at least about 1 hour, and lysed. The cell lysate is resolved by SDS PAGE, then transferred to nitrocellulose and probed with antibodies specific for β-catenin.

A plurality of assays may be run in parallel with different concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in binding.

Compounds of interest for screening include biologically active agents of numerous chemical classes, primarily organic molecules, although including in some instances inorganic molecules, organometallic molecules, immunoglobulins, chimeric frizzled proteins, frizzled related proteins, genetic sequences, etc. Also of interest are small organic molecules, which comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Compounds are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Molecules of interest as inhibitors of wnt include specific binding members that bind to, e.g. wnt, frizzled, wnt co-receptors, and the like. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). Inhibitors useful in the methods of the invention include analogs, derivatives and fragments of the original specific binding member.

In a preferred embodiment, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Fruend's, Fruend's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the brain tumor protein target, a superior immune response may be elicited if the polypeptide is joined to a carrier protein, such as ovalbumin, BSA or KLH. Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. Humanized, chimeric, or xenogenic human antibodies, which produce less of an immune response when administered to humans, are preferred for use in the present invention.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

In one embodiment of the invention, the protective agent, or a pharmaceutical composition comprising the protective agent, is provided in an amount effective to detectably inhibit the binding of extracellular wnt to frizzled present on the surface of said stem cell. In one embodiment, the protective agent is selected from: soluble FZD CRD; antibodies to FZD; secreted frizzled-related proteins (sFRPs), antibodies to Wnt; antibodies LRP5/6; antibodies to Kremen; Dkk proteins, Soggy protein, Wise; fusions proteins comprising any of the above; derivatives of any of the above; variants of any of the above; and biologically active fragments of any of the above. In another embodiment, the protective agent is selected from FZD8 CRD, FZD CRD-IgG fusion proteins, SFRP-1, SFRP-2, SFRP-3, SFRP-4, SFRP-5, Dkk-1, Dkk-2, Dkk-3, Dkk-4, Soggy, Wise, antibodies to wnt 3A, antibodies to wnt 2B; antibodies to wnt 10B and antibodies to wnt 5A.

Anti-proliferative agents: agents that act to reduce cellular proliferation are known in the art and widely used. Such agents include alkylating agents, such as nitrogen mustards, e.g. mechlorethamine, cyclophosphamide, melphalan (L-sarcolysin), etc.; and nitrosoureas, e.g. carmustine (BCNU), lomustine (CCNU), semustine (methyl-CCNU), streptozocin, chlorozotocin, etc. Such agents are used in the treatment of cancer, as well as being immunosuppressants and anti-inflammatory agents.

Other natural products include azathioprine; brequinar; alkaloids and synthetic or semi-synthetic derivatives thereof, e.g. vincristine, vinblastine, vinorelbine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithrmycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other chemotherapeutic agents include metal complexes, e.g. cisplatin (cis-DDP), carboplatin, etc.; ureas, e.g. hydroxyurea; and hydrazines, e.g. N-methylhydrazine. Other anti-proliferative agents of interest include immunosuppressants, e.g. mycophenolic acid, thalidomide, desoxyspergualin, azasporine, leflunomide, mizoribine, azaspirane (SKF 105685), etc., taxols, e.g. paclitaxel, etc.

Retinoids, e.g. vitamin A, 13-cis-retinoic acid, trans-retinoic acid, isotretinoin, etc.; carotenoids, e.g. beta-carotene, vitamin D, etc. Retinoids regulate epithelial cell differentiation and proliferation, and are used in both treatment and prophylaxis of epithelial hyperproliferative disorders.

In particular, antimetabolites and inhibitors of DNA topoisomerase are relatively ineffective against quiescent cells. Irinotecan (CPT-11) is a topoisomerase I inhibitor. CPT-11 finds use as a therapeutic agent, e.g. in the treatment of solid tumors, such as colon cancer, sarcomas, non-small cell lung carcinoma, ovarian and endometrial carcinomas, adenocarcinomas, mesotheliomas, etc. Other topoisomerase inhibitors of interest include doxorubicin and carboplatinum, which inhibit type II topoisomerase.

Antimetabolite agents include pyrimidines, e.g. cytarabine (CYTOSAR-U), cytosine arabinoside, fluorouracil (5-FU), floxuridine (FUdR), etc.; purines, e.g. thioguanine (6-thioguanine), mercaptopurine (6-MP), pentostatin, fluorouracil (5-FU) etc.; and folic acid analogs, e.g. methotrexate, 10-propargyl-5,8-dideazafolate (PDDF, CB3717), 5,8-dideazatetrahydrofolic acid (DDATHF), leucovorin, etc. Methotrexate is widely used as an immunosuppressant, particularly with allogeneic organ transplants, as well as in the treatment of other hyperproliferative disorders. Leucovorin is useful as an anti-infective drug.

Pharmaceutical Formulations: The wnt inhibitor, and the anti-proliferative agent can be incorporated into a variety of formulations for therapeutic administration. The wnt inhibitor, and the anti-proliferative agent can be delivered simultaneously, or within a short period of time, by the same or by different routes. In one embodiment of the invention, a co-formulation is used, where the two components are combined in a single suspension. In another embodiment, the two are separately formulated. Also included are formulations of wnt, or other agents that specifically block the inhibitor for use in chasing the inhibitor, following treatment with an anti-proliferative drug.

The active agents may be administered by any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intraarterial, intramuscular, intrasternal, intratendinous, intraspinal, intracranial, intrathoracic, infusion techniques or intraperitoneally.

The wnt inhibitors are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration can be achieved in various ways, usually by oral administration. The agent may be systemic after administration or may be localized by virtue of the formulation, or by the use of an implant that acts to retain the active dose at the site of implantation.

In pharmaceutical dosage forms, the wnt inhibitor and/or other compounds may be administered in the form of their pharmaceutically acceptable salts, or they may also be used alone or in appropriate association, as well as in combination with other pharmaceutically active compounds. The agents may be combined to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of glutenase in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Those of skill will readily appreciate that dose levels can vary as a function of the specific enzyme, the severity of the symptoms and the susceptibility of the subject to side effects. Some of the agents will be more potent than others. Preferred dosages for a given agent are readily determinable by those of skill in the art by a variety of means. A preferred means is to measure the physiological potency of a given compound.

### Therapeutic Methods

The dosage regimen for increasing stem cell survival following chemotherapy is based on a variety of factors, including the type of injury, the age, weight, sex, medical condition of the individual, the severity of the condition, the route of administration, and the particular compound employed. Thus, the dosage regimen may vary widely, but can be determined routinely by a physician using standard methods. Dosage levels of the order of between 0.1 ng/kg and 10 mg/kg body weight of the active agents per body weight are useful for all methods of use disclosed herein.

The methods find use in conditions where an antiproliferative agent is administered, and where it is desirable to spare normal stem cells that are otherwise killed by the anti-proliferative agent. The patient is typically mammalian, and may be primate, including human, may be used for veterinary purposes, e.g. canines, felines, ovines, equines, etc., or may be used in animal models for disease, e.g. murines, including rats and mice, lagomorphs, and the like. Conditions treated by anti-proliferative agents include treatment of autoimmune disease; antimicrobial treatments, particularly treatment of parasites and other eukaryotic microbes; and particularly, for the treatment of cancers. The treatment of cancer with anti-proliferative agents is well-known in the art, and need not be repeated herein. Of particular interest is the treatment of colon cancers, breast cancers, lung cancer, skin cancer, leukemias and lymphomas.

In the methods of the invention, an effective dose of a wnt inhibitor will render stem cells, e.g. hematopoietic stem cells, bone marrow mesenchymal stem cells, neural stem cells, gut stem cells, etc., quiescent for a period of time, without permanent damage to the stem cell viability. Typically a dose will be effective for at least the period of time during which an anti-proliferative agent is being administered, usually at least about 12 hours, more usually at least about 1 day, and frequently for a period of about 2 days, about 3 days, or more, usually not more than about 2 weeks, more usually not more than about 7 days. The therapy is administered for 1 to 6 times per day at dosages as described below. In all of these embodiments, the protective compounds of the invention can be administered prior to, simultaneously with, or subsequent to chemotherapeutic exposure. For example the compounds may be administered about 3 days prior, 2 days prior, or 1 day prior to chemotherapy.

Optionally, after a period of time that is effective for action of the anti-proliferative agent, a dose of wnt polypeptide or wnt mimetic is administered to the patient, in a dose that competitively blocks the wnt inhibitor, allowing normal stem cell proliferation to resume. The methods may be combined with various supportive therapy used in the art, e.g. administration of erythropoietin, GM-CSF, G-CSF, etc., usually after resumption of stem cell proliferation; transfer of blood cells including stem and progenitor cells, red cells, etc.

In another embodiment of the invention, a subject undergoes repeated cycles of treatment according to the method of this invention. Preferably, a subsequent treatment cycle commences only after the administration of the compounds of the invention has been terminated and the subject's blood cell counts (e.g., white blood cell count) have returned to a therapeutically acceptable level, permitting the repeated chemotherapy.

Kits are provided for increasing stem cell survival following chemotherapy, wherein the kits comprise an effective amount of the protective agent for increasing stem cell survival following chemotherapy, and instructions for using the amount effective of active agent as a therapeutic. Optionally, the kit further comprises a wnt or other quenching molecule in composition suitable for administering to chase the protecting agent at the conclusion of chemotherapy. Quenching molecules are any agent that specifically inactivates the protecting agent, either competitively or non-competively.

In a preferred embodiment, the kit further comprises a pharmaceutically acceptable carrier, such as those adjuvants described above. In another preferred embodiment, the kit further comprises a means for delivery of the active agent to a patient. Such devices include, but are not limited to syringes, matrical or micellar solutions, bandages, wound dressings, aerosol sprays, lipid foams, transdermal patches, topical administrative agents, polyethylene glycol polymers, carboxymethyl cellulose preparations, crystalloid preparations (e.g., saline, Ringer's lactate solution, phosphate-buffered saline, etc.), viscoelastics, polyethylene glycols, and polypropylene glycols. The means for delivery may either contain the effective amount of the active agents, or may be separate from the compounds, which are then applied to the means for delivery at the time of use.

The protective agent may be formulated with an anti-proliferative agent, including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound that is capable of destroying proliferating cells.

In one embodiment, the kit comprises a protective agent that blocks extracellular wnt signaling and instructions for administering to a patient said protective agent in an amount effective to detectably inhibit the binding of extracellular wnt to frizzled present on the surface of said stem cell as a therapeutic. The kit may further comprise a pharmaceutically acceptable carrier with which to admix said protective agent; and may comprise a means for delivery of the protective agent to a patient. The kit may further comprise a chemotherapeutic agent and instructions for administering to a patient said chemotherapeutic agent in conjunction with said protective agent in a therapeutic regime. The kit may further comprise a wnt polypeptide or a wnt mimetic and instructions for administering to a patient said wnt polypeptide or said wnt mimetic in an amount effective to competitively blocks the protective agent and allow normal stem cell proliferation to resume in a therapeutic regime.

### EXPERIMENTAL

### Example 1

### Assessment of Stem Cell Dependence on Wnt Signaling

HSCs in their normal microenvironment activate a LEF-1/TCF reporter, which indicates that HSCs respond to Wnt signaling in vivo. To demonstrate the physiological significance of this pathway for HSC proliferation, it is shown herein that the ectopic expression of axin or a frizzled ligand-binding domain, both of which are inhibitors of the Wnt signaling pathway, led to inhibition of HSC growth in vitro and reduced reconstitution in vivo. Furthermore, activation of Wnt signaling in HSCs induces increased expression of HoxB4 and Notch1, genes previously implicated in self-renewal of HSCs. It can be concluded that the Wnt signaling pathway is critical for normal HSC homeostasis in vitro and in vivo.

β-catenin expression leads to self-renewal of HSCs in vitro. We first determined the effects of activating downstream components of the Wnt pathway on HSC function. We activated Wnt signaling in HSCs sorted via fluorescence-activated cell sorting (FACS) (c-Kit⁺ Thy-1.1^{lo} Lin^{-/lo} Sca-1⁺ (KTLS) cells) by retrovirally transducing them with constitutively active β-catenin. Successful transduction of HSCs with retroviruses requires induction of cell cycle entry through the use of multiple growth factors, which can promote differentiation of stem cells in vitro. To minimize the pro-differentiation stimuli encountered by HSCs during infection before experiments of interest, we used HSCs from H2K-BCL-2 transgenic mice, which proliferate in the presence of steel factor (SLF) alone. Sorted BCL-2 transgenic HSCs were infected with retroviruses encoding either β-catenin-IRES-GFP (β-catenin, internal ribosome entry site and green fluorescent protein) or IRES-GFP alone, and GFP expression was detected in 45-55% of HSCs, which persisted for the entire in vitro culture period. GFP-positive (GFP⁺) HSCs were sorted to determine growth kinetics in vitro and the ability to reconstitute the immune system in vivo.

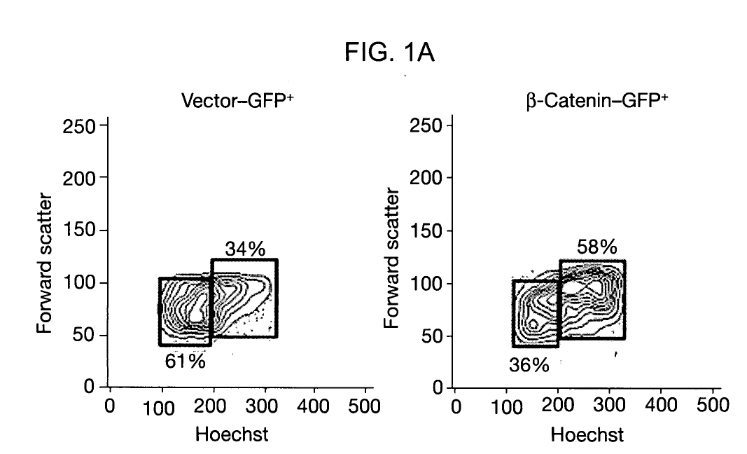

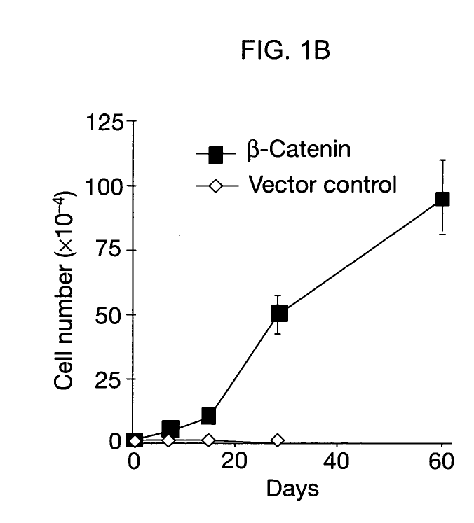

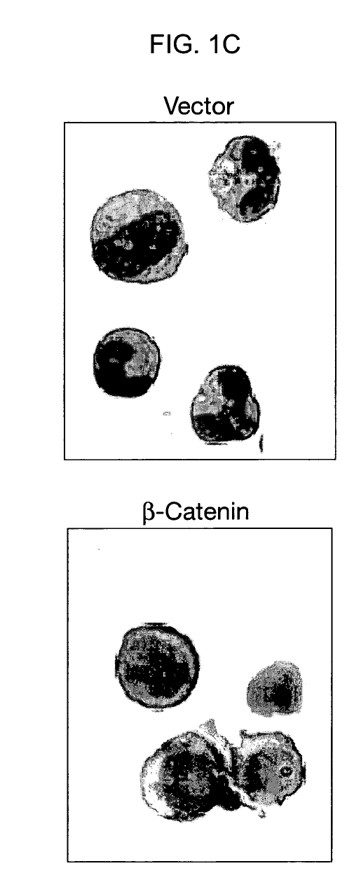

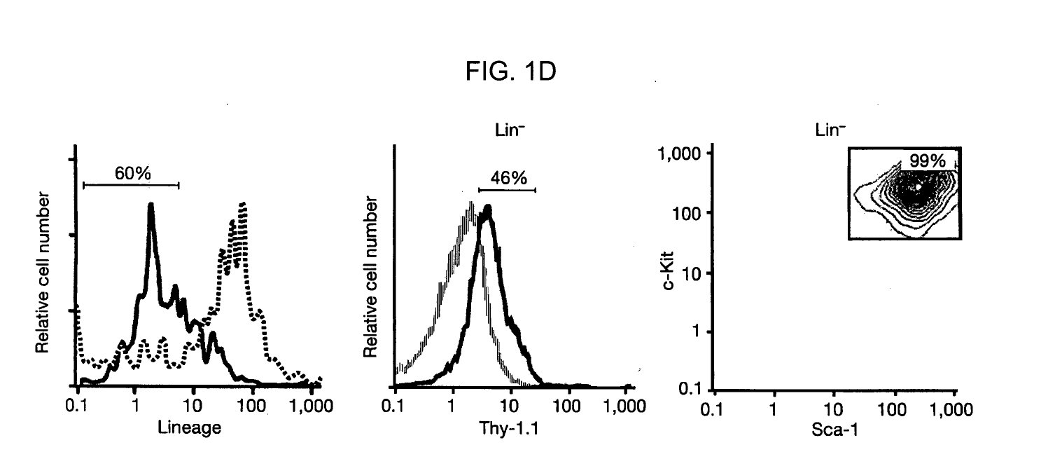

Short-term growth characteristics of HSCs expressing β-catenin or control vector were determined by cell cycle analysis. In FIG. 1A, whereas 34% of the HSCs infected with control vector were in S/G2/M phases of the cell cycle, 58% of the HSCs expressing activated β-catenin were in the same phases of the cell cycle. To determine whether activated Wnt signaling increased long-term growth, HSCs expressing β-catenin were grown in vitro in serum-free medium in the presence or absence of growth factors. Medium containing limiting amounts of SLF allowed the growth of β-catenin-transduced HSCs consistently for at least 8 weeks (FIG. 1b). During this period the GFP⁺ cells underwent eight to nine population doublings to generate at least 100 times the number of input cells. In contrast, HSCs infected with control vector showed minimal growth beyond a two-week period. On complete withdrawal of SLF during long-term culture, β-catenin-infected HSCs grew for at least 4 weeks, and in some experiments could be maintained and passaged for as long as 1-2 months. In contrast the control transduced HSCs did not survive beyond 48 h.

To determine whether growth in response to activated β-catenin was accompanied by differentiation, the morphological characteristics of these cells were analyzed at the end of a two-week period. This time point was chosen to be able to compare the differentiation status of control and β-catenin-transduced HSCs, as the lifespan of HSCs transduced with control vector was limited. Cells infected with control vector were found to have a myelo-monocytic appearance. In contrast, 65-75% of the β-catenin-transduced HSCs had a high nuclear to cytoplasm ratio (FIG. 1C). Consistent with this, although most (75-80%) of the HSCs infected with control vector were positive for lineage markers (FIG. 1D), only 5-10% of cells infected with β-catenin expressed high levels of lineage markers (predominantly Mac-1, an integrin expressed on fetal HSCs and regenerating HSCs). In fact, 60% of HSCs infected with β-catenin were lineage-negative and expressed high levels of c-Kit and Sca-1 and almost half of these also expressed low levels of Thy-1.1. Thus, at least 30% of the cells in β-catenin-transduced cultures had retained the phenotype of HSCs; that is, c-Kit⁺ Thy1.1^{lo} Lin⁻ Sca-1⁺ (KTLS cells). This indicated that the expression of activated β-catenin maintained hematopoietic stem cells in an immature state, while simultaneously allowing these cells to proliferate, thus expanding the HSC pool 20- to 48-fold on the basis of the total numbers of cells generated.

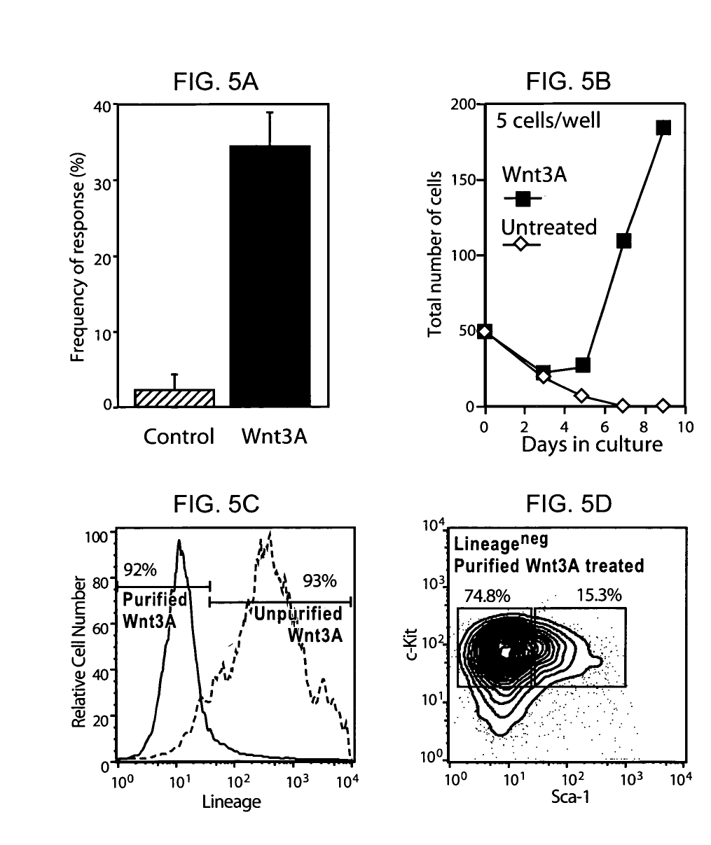

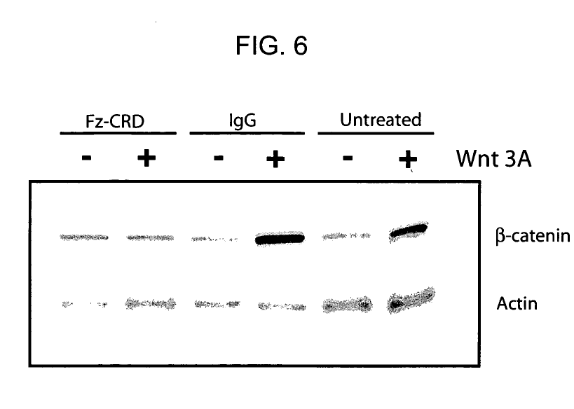

Without wishing to be bound by theory, we believe that the expansion of HSCs owing to activated β-catenin reflects upstream Wnt signals. It was demonstrated that purified Wnt3a causes self-renewal in both BCL-2 transgenic and wild-type HSCs (FIGS. 5-6). Specifically, singly plated HSCs generate six-fold or more numbers of progeny in the presence of Wnt3a compared with control conditions. These daughter cells not only maintain an immature phenotype, but also display a 5- to 50-fold expansion of HSC function as determined by transplantation analysis of the progeny of single HSCs after expansion in vitro.

Based on the numbers of cells seeded after beta-catenin infection (10,000) and the increase in numbers over an eight week period (960,000), expression of activated beta-catenin in HSC typically led to at least a 20- to 48-fold expansion of cells with a stem cell phenotype (30% of 960,000=288,000, an underestimate as at least some of the 10,000 initial cells probably neither survive nor respond).

The data using limited dilution transplants allowed us to conclude that significant functional expansion of HSCs occurs in the presence of beta-catenin. Since all of the mice transplanted with 125 beta-catenin transduced HSCs were successfully reconstituted, we estimate based on efficiency of engraftment (10% KTLS cells can reconstitute the marrow) that each transplant must have contained at least 10 HSCs/125 cells (˜10%) and likely much more since the reconstitution observed was at a high level. In a representative experiment carried out for 1 week we observed that 6,000 HSCs plated result in 48,000 cells. Based on the fact that 10% of this expanded population retain HSC activity (4,800), and that 10% of the plated HSCs would read out functionally (600) this suggests at least an 8-fold and up to an 80-fold (if 100% of cultured cells retained HSC activity) expansion of HSC function in the presence of activated beta-catenin. However, based on the fact that there is significant cell death initially, as well as the fact that cycling cells are far more inefficient at transplanting in vivo (˜1/50 cells or 2% read out functionally), the lower estimate of 8-fold is very likely an underestimate of the expansion that actually occurred. Based on the proliferation observed in cultures carried out for a longer period of time (2 months, FIG. 1), we estimate that a 96-960 fold functional expansion of HSCs occured in long term cultures.

Wnt3A induces proliferation of wild type HSCs in vitro. Purified Wnt protein can regulate HSC self-renewal in the same manner as β-catenin in BCL-2 transgenic HSCs. To ensure that this response was not dependent on BCL-2 over-expression, we specifically tested whether wild type HSCs respond in a similar manner to purified Wnt3A as well. Over a period of days, HSCs plated at 1-20 cells per well, responded extremely robustly to Wnt3A in contrast to control conditions (e.g. 184 cells versus 0 when plated at 5 cells/well) (FIG. 5). The average frequency of cells that responded to Wnt3A over 3 independent experiments was 17-fold more than the proliferation to control conditions (limiting dose of SLF) when plated at 10 cells/well. These data are representative of over 9 independent experiments utilizing different numbers of input cells (1-20 cells/well). Furthermore, the phenotypic characteristics of HSCs treated with purified or unpurified Wnt3A were dramatically different. After 7 days in culture, a majority of HSCs treated with purified Wnt3A were negative for lineage markers (solid line) while a majority treated with unpurified Wnt3A strongly upregulated lineage markers (dashed line) (C). Furthermore, a significant fraction of the lineage negative population expressed c-Kit and Sca-1 consistent with a HSC phenotype (D).

To test whether the cells treated with purified Wnt3A underwent self-renewal functionally, purified HSCs were plated as 1 cell or as 10 cells, treated with Wnt3A and each well containing proliferating cells transplanted individually into lethally irradiated recipient mice along with 300,000 Sca-1⁻ Bone Marrow cells (A). Analysis of peripheral blood (PB) from each transplanted mouse revealed multilineage reconstitution indicative of a HSC readout (B). Since the empirically observed frequency of reconstitution of resting HSCs is ˜10% and of cycling HSCs ˜2%, the observed frequency of reconstitution of 100% for 1 plated cells is consistent with Wnt3A inducing a 10- to 50-fold increase in HSC activity, a range similar to that seen with BCL-2 transgenic HSCs. Additionally in independent experiments wells plated with 10 cells as well as those plated with 5 cells also displayed 100% reconstitution efficiency consistent with increased self-renewal of cycling HSCs in response to Wnt3A. The facts that HSCs proliferated in response to Wnt3A in vitro, the increased maintenance of stem cell phenotypic characteristics and the functional increase in self-renewal occurs in both BCL-2 transgenic and in wild type mice, demonstrates that ectopic expression of BCL-2 is not essential for the responsiveness of HSCs to Wnt3A.

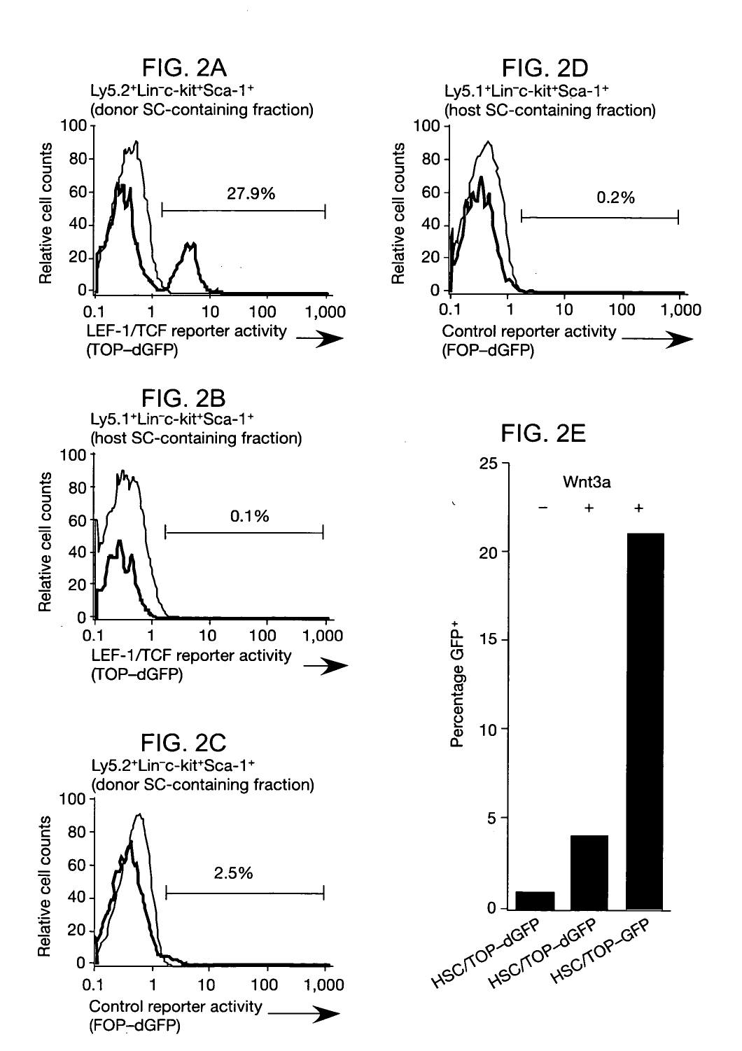

HSCs in vivo normally signal through LEF-1/TCF elements. To determine whether the Wnt signaling pathway is physiologically relevant to HSCs, we tested whether HSCs in vivo use signals associated with the Wnt/β-catenin pathway. HSCs were infected with LEF-1/TCF reporter driving expression of destabilized GFP (TOP-dGFP) or with control reporter with mutated LEF-1/TCF binding sites (FOP-dGFP), and then transplanted into lethally irradiated mice. Recipient bone marrow was examined after 14 weeks to determine whether donor HSCs demonstrated reporter activity. In the example shown, donor-derived HSCs infected with TOP-DGFP expressed GFP in 28% of the cells (FIG. 2; range observed 4-28%, mean 11.8%), whereas HSCs from the recipient mouse were negative for GFP (range observed 2.3-3.2%, mean 2.7%). Moreover, HSCs transduced with the control reporter did not express GFP significantly, demonstrating that functional LEF-1/TCF binding sites were required for HSC expression of GFP (FIG. 2C). In all cases, no reporter activity was observed in the non-HSC myeloid progenitor fraction (FIG. 2, thin line).

As a control, we also tested whether the TOP-DGFP reporter was turned on in response to Wnt3a-mediated signaling in HSCs in vitro. Thus, HSCs transduced with either TOP-DGFP or FOP-DGFP were stimulated with Wnt3a, and the extent of GFP expression was monitored. As shown in FIG. 2E, Wnt3a-treated HSCs showed significant reporter activity, demonstrating that the reporter is turned on in response to Wnt stimulus, but not in control conditions. Increased reporter activity was observed when the reporter construct driving non-destabilized GFP was used. These data demonstrate that HSCs in their normal microenvironment respond to endogenous Wnt signaling during self-renewal and/or stimulation into cell cycle, and also support the interpretation that the Wnt3a stimulus that caused increased self-renewal signals through the canonical Wnt pathway.

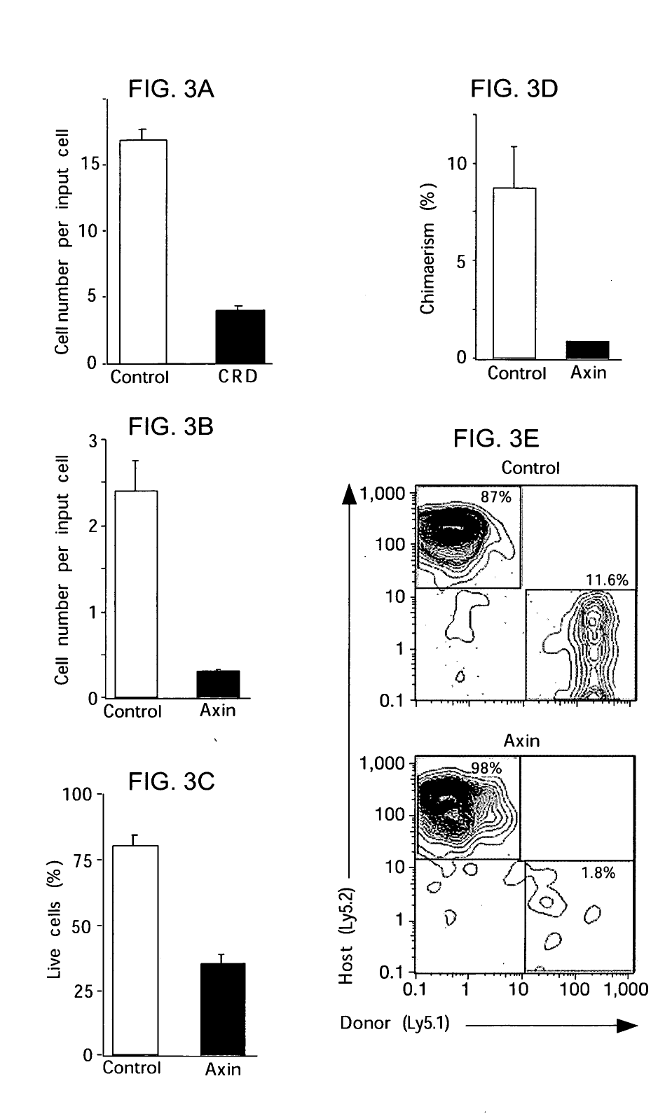

HSCs require intact Wnt signaling. To test whether Wnt signaling is required for normal HSC growth, we used a soluble form of the frizzled cysteine-rich domain (CRD) that inhibits the binding of Wnt proteins to the frizzled receptor (FIG. 6). Wild-type HSCs were incubated with growth factors in the presence of IgG-CRD domain fusion protein or control IgG, and cell proliferation was monitored. The presence of the CRD domain inhibited growth of HSCs fourfold compared with control conditions (FIG. 3A). This inhibition provides direct evidence of a Wnt signal modulating HSC survival and proliferation, as soluble CRD acts at the level of Wnt binding the frizzled molecules. Because only HSCs were present, the Wnt signal is probably derived from some or all of the HSCs in the cultures, and is required despite the presence of multiple other growth factors. These results can be interpreted to mean that all HSC mitoses are the result of Wnt signaling, even if the primary signals are not Wnt.

We also inhibited Wnt signaling through an independent inhibitor by ectopically expressing axin in HSCs. Axin increases β-catenin degradation and acts as an intracellular inhibitor of Wnt signaling. Live axin-infected wild-type HSCs were re-sorted 48 h after infection and plated in limiting numbers to assay growth in response to a combination of growth factors. Although control-infected cells proliferated 2.3-fold over 60 h, axin-infected cells showed a sevenfold reduction in the total growth response (FIG. 3b). Axin had an inhibitory effect on growth of BCL-2 transgenic HSCs as well, which suggests that expression of BCL-2 cannot protect cells from loss of Wnt signaling. To determine whether axin expression had an effect on cell survival, GFP⁺ cells were analyzed at the end of the infection period using propidium iodide exclusion. Whereas 80% of the control-infected cells were negative for propidium iodide, only 38% of axin-infected HSCs were negative for propidium iodide, indicating that axin expression has significant effect on cell survival by blocking β-catenin function.

To determine whether Wnt signaling is required for hematopoietic stem cell responses in vivo, we injected axin- or control-transduced viable HSCs into lethally irradiated mice and analyzed the level of reconstitution after 10 weeks. Mice transplanted with control-infected HSCs displayed on average sevenfold greater chimerism (reconstitution range 5-11.6%) than mice transplanted with axin-infected HSCs (reconstitution range 0-1.8%) (FIG. 3E). A representative example of contribution from axin- or vector-infected HSCs in transplanted mice is shown in FIG. 3d. These data show that inhibition of the Wnt pathway reduces reconstitution, suggesting that Wnt signaling is required for normal development of HSCs in vivo. This finding, together with the finding that HSCs respond to Wnt signaling in vivo (FIG. 2), indicates that Wnt/β-catenin signaling is an important physiological mediator of HSC-derived hematopoiesis.

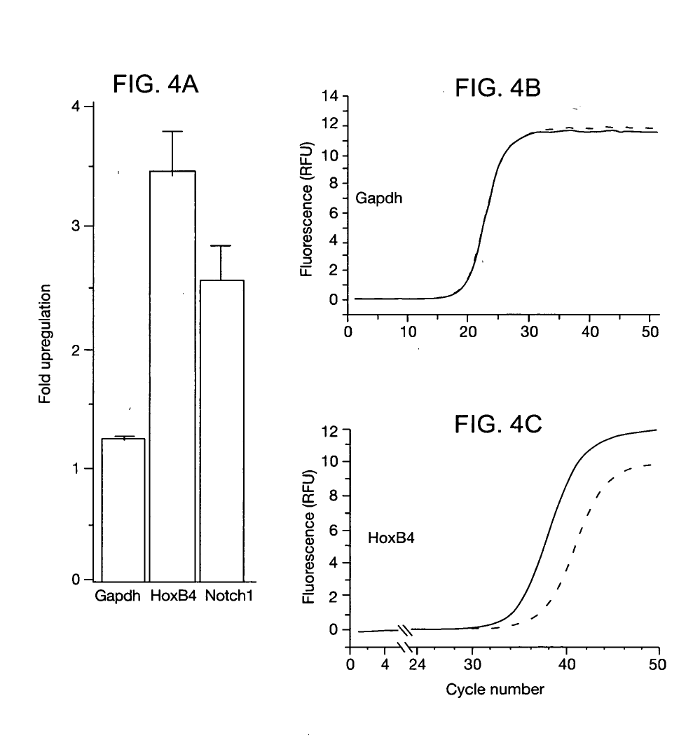

β-catenin upregulates HoxB4 and Notch1 in HSCs. We wished to determine whether Wnt signaling might be regulating HSC self-renewal by upregulating genes previously implicated in HSC self-renewal. To this end we tested upregulation of HoxB4 and Notch1. By using real-time polymerase chain reaction (PCR) analysis on HSCs infected with either β-catenin or control vector, we found that HoxB4 was upregulated an average of 3.5-fold and Notch1 was upregulated 2.5-fold (FIG. 4a). In contrast, GADPH expression was not differentially regulated as a consequence of β-catenin expression, and was used as a control (FIG. 5b). These data show that genes so far identified as regulators of HSC self-renewal may be related and perhaps act in a molecular hierarchy.

The above data show that components of the Wnt signaling pathway can induce proliferation of purified KTLS bone marrow HSCs while significantly inhibiting their differentiation, thereby resulting in functional self-renewal. Expression of β-catenin in HSCs results in increased growth with significantly reduced differentiation in vitro for a period of at least many weeks. HSCs transduced with β-catenin give rise to sustained reconstitution of myeloid and lymphoid lineages in vivo, when transplanted in limiting numbers. Wnt signaling is required for the growth response of normal HSCs to other cytokines, as overexpression of axin leads to reduced stem cell growth both in vitro and in vivo. Furthermore, the inhibition of HSC growth with frizzled-CRD and the finding that Wnt3a causes expansion of HSCs supports the interpretation that the effects of β-catenin and axin reflect upstream Wnt activity. Finally, studies with HSCs containing a LEF-1/TCF reporter indicate that HSCs in vivo respond to endogenous Wnt stimulation. The expression of a number of Wnt proteins in the bone marrow and frizzled receptors in bone-marrow-derived progenitors and HSCs supports this possibility.

Most growth factors that act on HSCs in culture induce no or limited expansion or are unable to prevent differentiation. Thus, one of the most notable findings of our work is the induction of proliferation and the prevention of HSC differentiation by the Wnt signaling pathway. Other signals that increase proliferation of HSCs include Notch and sonic hedgehog. Moreover, the cyclin-dependent kinase inhibitor p21^{Cip1/Waf1} and the transcription factor HoxB4 have been shown to be involved in regulating self-renewal of HSCs. Notably, Wnt signaling has been shown to interact with many of these pathways in a variety of organisms, and the above data show that both HoxB4 and Notch1 are upregulated in response to Wnt signaling in HSCs.

These findings have important implications for human hematopoietic cell transplantation. Soluble Wnt3a protein induces proliferation of highly purified human bone marrow HSCs in the absence of any other growth factor. Induction of HSC growth by Wnt signaling may allow in vitro expansion of a patient's own or an allogenic donor's HSCs, and could provide an increased source of cells for future transplantation. Conversely, by inhibiting Wnt signaling, HSC can be arrested in a quiescent stage.

Materials and Methods

Mice. C57Bl/Ka Ly5.1, Thy-1.1 (wild-type and BCL-2), C57Bl/Ka Ly5.2, Thy-1.1, and AKR/J mice were used at 6-10 weeks of age. Mice were bred and maintained on acidified water in the animal care facility at Stanford and Duke University Medical Centers.

HSC isolation. We sorted HSCs from mouse bone marrow. All cell sorting and FACS analysis was carried out on a FACSVantage (Becton Dickinson) at the Stanford shared FACS facility and the Duke Cancer Center FACS facility. Cells were sorted and reanalyzed on the basis of expression of c-Kit, Sca-1, low levels of Thy-1.1, and low to negative levels of lineage markers (Lin).

Cell cycle analysis. Retrovirally transduced HSCs were collected from cultures and stained with Hoechst 3342 (Molecular Probes) at 37° C. for 45 min in Hoechst medium. Cells were then washed and analyzed by Flow cytometry to determine the cell cycle profile of GFP⁺ cells.

Viral production and infection. Virus was produced by triple transfection of 293T cells with murine stem cell virus constructs along with gag-pol and vesicular stomatitis virus G glycoprotein constructs. Viral supernatant was collected for three days and concentrated 100-fold by ultracentrifugation at 50,000 g. For viral infection, 10,000 HSCs were sorted into wells of a 96-well plate and cultured overnight in the presence of SLF (30 ng ml⁻¹) for BCL-2 transgenic HSCs, or SLF (30 ng ml⁻¹) plus TPO (30 ng ml⁻¹) for wild-type HSCs. After 12 h, concentrated retroviral supernatant was added to the cells at a 1:1 ratio. Cells were then incubated at 32° C. for 12 h and 37 C. for 36 h before GFP⁺ cells were sorted for in vitro and in vivo assays. Lentiviruses used were produced as previously described. Briefly, 293T cells were transfected with the transfer vector plasmid, the VSV-G envelope-encoding plasmid pMD.G, and the packaging plasmid CMVΔR8.74. The supernatant was collected and concentrated by ultracentrifugation. All cytokines were purchased from R&D systems.

In vitro HSC proliferation assays. Freshly purified or virally transduced HSCs were plated at 1 to 20 cells per well in Terasaki plates. Cells were sorted into wells containing serum-free medium (X-vivo15, BioWhittaker) supplemented with 5×10⁻⁵ M 2-mercaptoethanol and the indicated growth factors. Proliferation was monitored by counting the number of cells in each well at defined intervals. For longer-term cultures, transduced HSCs were plated in 96-well plates in the absence or presence of SLF (1 ng ml⁻¹), and the number of cells generated was monitored by cell counting at defined intervals. For inhibition of growth by CRD or axin, cells were cultured in the presence of mitogenic factors (SLF (30 ng ml⁻¹), Flt-3L (30 ng ml⁻¹), interleukin-6 (10 ng ml⁻¹)).

In vivo analysis of HSC function. Virally transduced HSCs were cultured in vitro and injected retro-orbitally into groups of 4-6 congenic recipient mice irradiated with 9.5 Gy using a 200-kV X-ray machine, along with 300,000 rescuing host total bone marrow or Sca-1-depleted bone marrow cells. Host mice were given antibiotic water after irradiation. Transplanted mice were bled at regular periods to determine the percentage of the hematopoietic compartment contributed by donor cells. Donor and host cells were distinguished by allelic expression of CD45 (Ly5) or expression of the BCL-2 transgene.

Lentiviral reporter assays. The enhanced GFP (eGFP) or the d2-eGFP gene (destabilized, half-life of 2 h; Clontech) was cloned downstream of a LEF-1/TCF-responsive promoter, containing three LEF-1/TCF binding motifs and a TATA box. This cassette was then cloned into a self-inactivating lentiviral vector plasmid, and virus was produced as described above.

For in vivo assays, HSCs were transduced with reporter lentiviruses and cultured in X-Vivo15 with glutamate, 5×10⁻⁵ M 2-mercaptoethanol, and a cocktail of cytokines including 10 ng ml⁻¹ interleukin-11, 10 ng ml⁻¹ TPO, 50 ng ml⁻¹ SCF, 50 ng ml⁻ Flt-3L. Cells were incubated at 37° C. for 6 h overnight and transplanted into lethally irradiated congenic recipients. Lethally irradiated mice received 500 transduced HSCs along with rescue bone marrow. For analysis, hematopoietic progenitor cells were analyzed for reporter activation 14-24 weeks after transplantation.

For in vitro assays, purified HSCs were sorted directly into medium (IMDM/10% FBS plus interleukin-11, TPO, SCF and Flt-3L, as above) and plated at 500-1,000 cells per well in 96-well plates. Individual wells were transduced with the appropriate lentiviral reporter and stimulated with or without purified Wnt3a (about 100 ng ml⁻¹). Cells were collected 5 days later, stained with propidium iodide to exclude non-viable cells, and analyzed for GFP expression.

Real-time PCR analysis. A total of 75,000 HSCs cultured in 96-well plates containing X-Vivo15, 5×10⁻⁵ M 2-mercaptoethanol and 100 ng ml⁻¹ SLF were infected with either β-catenin or control lentiviruses. After two days in culture, transduced cells were isolated on the basis of GFP expression. RNA was prepared using Trizol (Invitrogen) and linearly amplified using a modified Eberwine synthesis. Each amplified RNA was converted to the first strand and analyzed for differential gene expression by real-time PCR. Complementary DNAs were mixed with FastStart Master SYBR Green polymerase mix (Roche), primers and real-time PCR was performed using a LightCycler (Roche).

### Example 2

### Analysis of Human Stem Cell Viability in an Animal Model

A SCID-hu animal model is set up for human bone marrow. The human HSC are tested after induction of quiescence for the presence of non-proliferating cells; and for the resumption of normal hematopoiesis after the quiescent period. The cells are then tested for resistance to killing by anti-proliferative agents that target proliferating cells.

Scid-hu bone marrow model. Human fetal femurs and tibias (1-2 cm) at 17-22 gestational week (g.w.), which are known to be active in hematopoiesis, are cut along a longitudinal axis so that bone cortex as well as intramedullary regions is exposed. These fragments are then surgically implanted subcutaneously into SCID mice. Homozygous CB-17 scid/scid mice are bred, treated with antibiotics as described (McCune et al., Science (1988) 241:1632), and used when 6-8 weeks old. Methoxyflurane anesthesia is applied during all operative procedures. Hematoxylin-eosin stained tissue sections are prepared from bone grafts 2 weeks and 8 weeks after implantation. The tissues are fixed in 20% formalin, decalcified with EDTA (1.7 mM) in HCl solution, paraffin embedded, and 4 μm sections are cut and stained with hematoxylin and eosin. Grafts are removed at varying intervals after implantation and analyzed for the presence of human hematopoietic activity.

The cell suspensions are prepared from implanted or normal bone marrow tissues, treated with 0.83% of ammonium chloride for 5-10 min at room temperature to lyse red blood cells, and washed with PBS. The cells are incubated with either biotinylated-MEM-43, biotinylated-Ly5.1, or biotinylated control antibodies for 45 min on ice, washed through a fetal bovine serum (FBS) cushion, and then stained with fluorescein conjugated (FITC-) avidin (Caltag Laboratories Inc.) for 45 min. Before flow cytometry, propidium iodide (PI) is added at final concentration of 10 μg/ml to gate out dead cells. Forward and side scattering patterns of the MEM-43 positive cells is obtained by four parameter flow cytometry using a single laser FACScan (Becton Dickinson Immunocytometry Systems).

At 4-5 weeks, active hematopoiesis is observed at many sites within the engrafted bones. After 6-8 weeks, most of the grafts looked similar to normal human fetal bone marrow associated with lymphopoiesis, myelopoiesis, erythropoiesis, and megakaryocytopoiesis in a high degree of cellularity. The yield of the cells from the grafts 4-16 weeks after implantation is approximately 10% of the input. Wright-Giemsa staining of these cells on cytospin preparations also reveals the typical morphology of lymphoid, myeloid or erythroid cells at different maturational stages. These signs of active hematopoiesis are observed in more than 90% of the bone grafts and continue to 16 weeks after implantation.

The human origin of hematopoietic cells within the grafts is confirmed by flow cytometry with either MEM-43 (an antibody specific for a common antigen of human cells) or Ly5.1 (reactive with mouse pan-leukocyte antigen). The replacement of the human bone marrow with mouse hematopoietic cells is observed in some of the grafts incubated in vivo for over 20 weeks.

The characteristics of the hematopoietic cell populations in the bone marrow are analyzed by light scattering profiles using flow cytometry. Four distinctive clusters of hematopoietic cells, i.e., lymphoid (Rl), blastoid (R2), myeloid (R3), and mature granulocyte (R4) populations are revealed in normal fetal bone marrow by forward and side scattering distributions. Similar analyses with MEM-43 positive human cells recovered from the bone implants at various different time points after implantation are carried out. Cells recovered 2 weeks after implantation do not show clear cluster formation, indicating that these cells are of non-hematopoietic origin, while the human cells from grafts incubated longer than 4 weeks showed scattering profiles that are similar to those of normal fetal bone marrow cells. Thus, the kinetics of the appearance of human hematopoietic cells in the implanted bone detected by scatter analyses is found to be in accord with the histological observations.

The cell surface phenotypes of the nucleated hematopoietic cells in the grafts can be further analyzed with various antibodies specific for human lineage markers. About 80% of the cells in the lymphoid (Rl) region are B cells, positive for both CD10 and CD19. When stained for surface immunoglobulin, about 20% express IgM and about 4% express IgD as well. The ratio of cells with either κ or λ light chains was similar to that in normal bone marrow, suggesting that these B cells are not products of a monoclonal expansion. A small number (<5%) of human T-lineage cells detected by CD7 antibody are found in this region. Approximately 60% of the cells in the myeloid (R3) region are found to express the CD15 antigen, specific for myelomonocytic cells, indicating that the major population of the cells in this region was the immature forms of myelomonocytic cells. Over 80% of the cells in the R4 region are also positive for this marker and the light scattering profile indicated that they are mature forms of granulocytes. The cell population in the blastoid (R2) region is a mixed population of CD10⁺ CD19⁺ cells, CD15⁺ cells, and cells lacking these markers. Furthermore, as observed in normal fetal bone marrow, a significant (5-10%) number of cells in the R1 and R2 regions express CD34, a marker for bone marrow progenitor cells. Taken together, the cellular composition in each cluster in the implanted human bone marrow is found to be similar to those of normal fetal bone marrow.

The level of human erythropoietic activity is analyzed with antibodies specific for human glycophorin A (GPA). Flow cytometric analysis of human glycophorin A (GPA) expression in bone marrow cells from the grafts is performed. The cell suspensions are prepared from the grafts without ammonium chloride treatment. The cells are stained with biotinylated-anti-human GPA antibodies, followed by FITC-avidin binding as described above. After final washing with PBS, the cells are fixed in 2.5% paraformaldehyde in PBS, and then incubated with Pl at the final concentration of 1 μg/ml to stain nuclear DNA.

Human progenitor cells with self-renewal and multi-lineage capacity are functionally maintained when human bone grafts are implanted into SCID mice. Kinetics of progenitor cell activities by colony forming assay in culture are examined.

The total number of colonies per graft is obtained by calculation based on the numbers of the colonies and the total cell number recovered. Bone grafts from different fetal donors are used for this experiment. CFU-GM and BFU-E are assayed by methylcellulose cultures, according to previously described methods. Briefly, the bone marrow cells are plated in 24 well plates at a concentration of 1-5×10⁴/ml in 0.25 ml cultures containing 1% methylcellulose in Iscove's modified Dulbecco's medium (Gibco Laboratories) with 20% FBS, 0.05 mM 2-mercaptoethanol, 200 mM L-glutamine, 0.8% lept-albumin, 0.08% NaHCO₃, and human recombinant erythropoietin (Amgen Biologicals) at the concentration of 2 u/ml, and 10% Mo conditioned media. The methylcellulose cultures are incubated at 37° C. in 7% CO₂ in air and are counted after 12 days to determine the number of colonies per well. CFU-C are characterized as having greater than 50 cells and consisted mainly of granulocytes and/or macrophages (CFU-GM) or multiple clusters of erythroid cells (BFU-E).

Finally, the presence of human cells in the peripheral circulation of SCID-hu mice with bone grafts is examined by FACS analysis, using the combination FITC-HLe1 antibody (the common human leukocyte antigen, CD45) and PE-W6/32 antibody (a monomorphic determinant of MHC-Class I). Human cells are detected at significant frequency in peripheral blood from the SCID-hu mice examined after 9 weeks of implantation.

To determine the effect of a wnt inhibitor on human progenitors in the bone marrow, CB-17 scid/scid mice in which are implanted human fetal bone from various long bones 8 to 10 weeks before, are treated at various dose levels with a CRD-Ig molecule, as described in Example 1. The animals are treated with an initial dose of the CRD-Ig; and after two days, cells are recovered from implanted bones. The number of proliferating stem cells is calculated by staining for human, CD34+, Thy-1+ cells; and staining with Ki67 (a nuclear protein expressed in proliferating cells during late G1-, S-, M-, and G2-phases of the cell cycle, but not in the G0 (quiescent) phase). The number of actively proliferating stem cells is normalized to a control animal.

To test the ability of the stem cells to resume normal proliferation, the animals are treated with various doses of Wnt3A protein, 3 days after the administration of the CRD-Ig. The wnt protein acts to wash out the inhibitor, and allows resumption of normal signaling. Two days later, the stem cells are again collected, and tested for the presence of proliferating cells as described above.

In order to establish the protection of stem cells from anti-proliferative agents, a dose of CRD-Ig that is sufficient to block proliferation, but which does not prevent resumption of proliferation following a wnt washout, is administered to the animals. 12 hours later, the animals are treated with a single dose of methotrexate at a dose equal to the LD₅₀ for HSC. A control animal is treated with methotrexate in the absence of the protective CRD-Ig. After 24 hours, the stem cell viability is calculated in the absence, or presence of the protective agent, in a colony assay as described above.

### Example 3

### Growth and Metastasis of Human Leukemia Cells in an Animal Host

A SCID-hu animal model is set up for human bone marrow, and is further tested by the addition of human leukemia cells. The human HSCs are tested after induction of quiescence for the presence of non-proliferating cells; and for the resumption of normal hematopoiesis after the quiescent period. The cells are then tested for resistance to killing by anti-proliferative agents that target the proliferating leukemia cells.

Patient samples. Bone marrow (BM) samples from myeloid leukemia patients, including acute myeloid leukemia and chronic myeloid leukemia in myeloid blast crisis, are obtained with informed consent. Mononuclear cells are isolated by Ficoll-Paque (Pharmacia) density sedimentation and are then cryopreserved in RPMI-1640 (GIBCO) containing 10% DMSO and 10% fetal bovine serum (FBS). After thawing, cells are washed with RPMI-1640 containing 10% FBS and used for flow cytometric analysis and for implantation.

SCID-hu mice. Homozygous C.B-17 scid/scid mice (SCID) are bred, treated with antibiotics, and used when 6-8 week old. Femurs and tibias of 19 to 23 gestational week human fetuses are cut into fragments and implanted subcutaneously into the mice. Cell suspensions prepared from thymus of individual fetal donors are analyzed for the HLA allotypes.

Injection of leukemia cells. After thawing, bone marrow cells of leukemia patients (0.4-2.0×10⁶ viable cells) are resuspended in 20 ml of RPMI-1640 containing 10% FBS and injected with a microliter syringe (Hamilton Co.) directly into the human fetal bone grafts. The bone grafts are implanted subcutaneously 6-8 weeks prior to the injection of leukemia cells. Combinations of bone and leukemia donors are selected to be disparate for commonly distributed HLA allotypes so that the origin of the cells in human bone implant can later be traced.

Antibodies. Mouse monoclonal antibodies against MHC class I antigens are directly conjugated with either FITC or PE. FITC-anti-LeuM1 (CD15), PE-anti-LeuM9 (CD33), PE-anti-Leu12 (CD19), FITC-anti-CALLA (CD10), and FITC-anti-HLe1 (CD45) are purchased.

Flow cytometry. Single cell suspensions are prepared from human bones and/or tumors by mincing tissues with scissors in cold RPMI-1640 containing 10% FBS. Cells are then treated with ammonium chloride to lyse red blood cells and stained by immunofluorescence for the indicated markers Cells from mouse peripheral blood and bone marrow are examined as well. Before analysis, propidium iodide is added at a final concentration of 10 μg/ml to selectively gate out dead cells. Multiparameter flow cytometry is performed using the FACScan system. Percent leukemia cells is calculated as the percentage of patient's HLA allotype positive cells per total human cells in the individual samples. In each experiment, isotype-matched antibodies are included as negative controls.

Histology. Cytocentrifuge slides are prepared and stained with the Wright-Giemsa stain.

Implantation Of Human Myeloid Leukemia Cells Into SCID-Hu Mice. Cryopreserved BM cells from leukemia patients are directly injected into human fetal bone fragments of SCID-hu mice. The growth of human leukemia cells in injected human BM, as well as mouse BM, is analyzed by flow cytometry 4-56 weeks after injection.

In order to establish the protection of stem cells from anti-proliferative agents, a dose of CRD-Ig that is sufficient to block proliferation, but which does not prevent resumption of proliferation following a wnt washout, is administered to the animals. Twelve hours later, the animals are treated with a single dose of CPT-11 at a dose equal to the LD₅₀ for HSC. A control animal is treated with CPT-11 in the absence of the protective CRD-Ig. After 24 hours, the stem cell viability is calculated in the absence, or presence of the protective agent, in a colony assay as described above. The number of viable tumor cells is similarly calculated.

### Example 4

Cells of human lung cancer cell lines are introduced intravenously into immunodeficient SCID mice implanted prior to inoculation with fragments of human fetal lung and human fetal bone marrow.

Mice and Tissues. Homozygous CB-17 scid/scid mice are used at the age of 6 to 8 weeks. Human fetal lungs at 18 to 22 gestational weeks are cut into fragments approximately 1 mm³ and surgically implanted into mouse mammary fat pads and under the kidney capsule. Human fetal femurs and tibias at the same gestational age are cut lengthwise and implanted subcutaneously into SCID mice. The resulting SCID-hu animals are used for experiments at 4 to 8 weeks post implantation.

Cell Lines. Small cell lung carcinomas (SCLC) cell lines N417 and H82 of variant subtype are obtained from National Cancer Institute, National Institutes of Health. Lung adenocarcinoma cell line A427 is obtained from ATCC. Cell lines are maintained in growth medium RPMI 1640 (N417 and H82) or DMEM (A427) supplemented with 10% fetal bovine serum.

Tumor cells are injected into SCID-hu mice intravenously via the lateral tail vein. Alternatively, cells are injected directly into human fetal tissues implanted subcutaneously into mice. Mice are examined twice a week for growth of tumors and sacrificed at or before the time when tumor volume reaches 5 cm³. Human lung implants, mouse lungs and other internal organs and tumors are examined histologically. Single cell suspensions are prepared from the aseptically removed and minced tumors by incubation for 1 hour at 37° C. in the presence of dispase and DNase. Cells are washed and used for intravenous injection or explanted in vitro to reestablish cell lines.

In order to establish the protection of stem cells from anti-proliferative agents, a dose of CRD-Ig that is sufficient to block proliferation, but which does not prevent resumption of proliferation following a wnt washout, is administered to the animals. Twelve hours later, the animals are treated with a single dose of CPT-11 at a dose equal to the LD₅₀ for HSC. A control animal is treated with CPT-11 in the absence of the protective CRD-Ig. After 24 hours, the stem cell viability is calculated in the absence, or presence of the protective agent, in a colony assay as described above. The number of viable tumor cells is similarly calculated.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method of inducing quiescence in normal stem cells, the method comprising:
contacting said normal stem cells with an effective dose of a protective agent that blocks the extracellular activation of the wnt pathway in said normal stem cells;
wherein said normal stem cells rendered quiescent.

2. The method according to claim 1, wherein said stem cells comprise hematopoietic stem cells.

3. The method according to claim 2, wherein said protective agent is administered in vivo to a patient.

4. The method according to claim 3, wherein said patient is suffering from cancer, and further comprising the step of administering to said patient an anti-proliferative agent concurrently with or following administration of said protective agent.

5. The method according to claim 4, wherein said anti-proliferative agent is selective for replicating cells.

6. The method according to claim 5, wherein said anti-proliferative agent is an anti-metabolite.

7. The method according to claim 6, wherein said anti-metabolite is selected from pyrimidines, such as cytarabine (CYTOSAR-U), cytosine arabinoside, fluorouracil (5-FU), or floxuridine (FUdR); purines, such as thioguanine (6-thioguanine), mercaptopurine (6-MP), pentostatin, or fluorouracil (5-FU); or folic acid analogs, such as methotrexate, 10-propargyl-5,8-dideazafolate (PDDF, CB3717), 5,8-dideazatetrahydrofolic acid (DDATHF), or leucovorin.

8. The method according to claim 5, wherein said anti-proliferative agent is a topoisomerase inhibitor.

9. The method according to claim 8, wherein said topoisomerase inhibitor is selected from irinotecan, doxorubicin or carboplatinum.

10. The method according to claim 7, further comprising the step of administering a wnt protein or wnt mimetic following said anti-proliferative agent in an amount effective to cause resumption of stem cell proliferation.

11. The method according to claim 1, wherein said protective agent binds to extracellular wnt, and inhibits the binding of said extracellular wnt to frizzled present on the surface of a stem cell.

12. The method according to claim 11, wherein said protective agent comprises at least a portion of a frizzled polypeptide.

13. The method according to claim 12, wherein said protective agent comprises a frizzled CRD fused to a plasma protein.

14. The method according to claim 13, wherein said plasma protein is a constant region of an immunoglobulin.

15. The method according to claim 11, wherein said protective agent comprises a soluble frizzled related polypeptide.

16. The method according to claim 1 wherein said protective agent comprises an immunoglobulin specific for wnt or frizzled.

17. A method for increasing stem cell survival in a patient to be administered a chemotherapy agent comprising the step of administrating to said patient at least one protective agent that blocks extracellular wnt signaling in an amount effective to detectably inhibit the binding of extracellular wnt to frizzled present on the surface of said stem cell, wherein said protective agent is administered prior to or simulataneously with said chemotherapy agent.

18. A pharmaceutical composition comprising:
at least one active protective agent that blocks extracellular wnt signaling in an amount effective to detectably inhibit the binding of extracellular wnt to frizzled present on the surface of said stem cell; a chemotherapeutic agent in a dose effective for chemotherapy; and a pharmaceutically acceptable carrier.

19. The method according to claim 1, wherein said protective agent is selected from: soluble FZD CRD; antibodies to FZD; secreted frizzled-related proteins (sFRPs), antibodies to Wnt; antibodies LRP5/6; antibodies to Kremen; Dkk proteins, Soggy protein, Wise; fusions proteins comprising any of the above; derivatives of any of the above; variants of any of the above; and biologically active fragments of any of the above.

20. The method according to claim 19, wherein said protective agent is selected from FZD8 CRD, FZD CRD-IgG fusion proteins, SFRP-1, SFRP-2, SFRP-3, SFRP-4, SFRP-5, Dkk-1, Dkk-2, Dkk-3, Dkk-4, Soggy, Wise, antibodies to wnt 3A, antibodies to wnt 2B; antibodies to wnt 10B and antibodies to wnt 5A.

21. A kit comprising a protective agent that blocks extracellular wnt signaling and instructions for administering to a patient said protective agent in an amount effective to detectably inhibit the binding of extracellular wnt to frizzled present on the surface of said stem cell as a therapeutic.

22. The kit according to claim 21, further comprising a pharmaceutically acceptable carrier with which to admix said protective agent.

23. The kit according to claim 22, further comprising means for delivery of the protective agent to a patient.

24. The kit according to claim 21, further comprising a chemotherapeutic agent and instructions for administering to a patient said chemotherapeutic agent in conjunction with said protective agent in a therapeutic regime.

25. The kit according to claim 21, further comprising a wnt polypeptide or a wnt mimetic and instructions for administering to a patient said wnt polypeptide or said wnt mimetic in an amount effective to competitively blocks the protective agent and allow normal stem cell proliferation to resume in a therapeutic regime.
